(19) United States
(12) Patent Application Publication
(10) Pub. No.: US 2011/0137273 A1
(43) Pub. Date: Jun. 9, 2011

(54) Fecal management system

(75) Inventors: Peter Müllejans, Aalsgaarde (DK); Michael Hansen, Gilleleje (DK); Troels Johansen, Nivaa (DK)

(73) Assignee: COLOPLAST A/S, Humlebaek (DK)

(21) Appl. No.: 12/672,537

(22) PCT Filed: Aug. 8, 2008
(86) PCT No.: PCT/DK2008/050196

§ 371 Date: Feb. 17, 2011

(30) Foreign Application Priority Data
Aug. 10, 2007 (DK) PA 2007 01139

(51) Int. Cl. A61F 5/44

(52) U.S. Cl. 604/355

(57) ABSTRACT

The present invention relates to a fecal management system for collecting fecal matter from the rectum. The system comprises a first fecal receiving bag and a second fecal receiving bag having an elongated section extending in a flow direction between a second proximal opening and a reservoir section, wherein the first fecal receiving bag and the second fecal receiving bag are connectable to each other. This provides a fecal management system that is adaptable to the user; e.g. the first fecal receiving bag can be produced as a compact and portable device, which allows for the user to move around as fecal matter will be collected in the first fecal receiving bag and thereby avoiding any embarrassing accidents; and when the user lies in bed the second fecal receiving bag can be coupled to the first fecal receiving bag, which allows for additional capacity for collecting e.g. nightly stools.

### FIELD OF THE INVENTION

The present invention relates to a fecal management system for collecting fecal matter from the rectum.

### BACKGROUND

Bedridden persons, for example persons being hospitalized, especially trauma patient who may not be fully conscious often suffer from fecal incontinence. Furthermore, people having an ostomy are often not capable of controlling their discharge. This may understandably result in very unhygienic situations and it is therefore desirable to control the fecal discharges and in particular prevent these discharges from entering the ambient environment, such as a person's bed.

To meet this need ostomy bags have been provided for ostomy users and fecal collecting bags for application around the rectum of anal incontinent persons.

However, it is not always desirable to change the bags every time they are full as this may cause skin irritation. Thus, alternatively it may be desirable to connect an additional collecting bag to the ostomy or fecal collecting bag, which may separately be disconnected when full allowing a new one to be connected.

U.S. Pat. No. 4,784,656 discloses a receptacle for collecting fecal matter from incontinent patients and in particular bedridden incontinent patients.

### SUMMARY OF THE INVENTION

The present invention relates to a fecal management system (hereinafter also referred to as the system) for collecting fecal matter from the rectum. The system comprises a first fecal receiving bag extending in a flow direction between a first proximal opening attachable around the rectum and a closable first distal opening. The system further comprises a second fecal receiving bag having an elongated section extending in the flow direction between a second proximal opening and a reservoir section. The reservoir section extends in the flow direction between the elongated section and a distal end. The first distal opening is connectable to the second proximal opening.

This provides a fecal management system that is adaptable to the user. The first fecal receiving bag can be produced as a compact and portable device. This allows for the user to move around as fecal matter will be collected in the first fecal receiving bag and thereby avoiding any embarrassing accidents. When the user lies in bed the second fecal receiving bag can be coupled to the first fecal receiving bag. This allows for additional capacity for collecting e.g. nightly stools. Moreover, the second fecal receiving bag is usually not intended to be carried around, thus, it may be provided with a much larger volume capacity than the first fecal receiving bag.

The elongated section allows for the reservoir section to be placed away from the user. Typically the elongated section has a length which allows for the reservoir section to be placed beyond the knee of the user, preferably it may also be extended to the side of the bed where the reservoir section can be hung so that a caregiver easily may observe the amount of fecal matter collected.

A closable second distal opening may be provided at the distal end of the reservoir section. This allows for the second fecal receiving bag to be emptied or allowing a caregiver to take samples.

The receiving bags can be made of many different materials suitable for containing and handling fecal matter in a leak proof and odor free manner. In one embodiment the receiving bag may be formed of extruded materials. However, materials suitable for extrusion tend to be elastic and have to be relative thick in order not transmit odor. Such thick materials will provide bulkier receiving bags that consequently are uncomfortably to wear or lie on. Thus, in one embodiment the first fecal receiving bag and the second fecal receiving bag is formed of a first sheet wall and a second sheet wall attached to each other along their periphery and additionally may be formed of non-elastic materials. By using sheet walls in order to form the receiving bags it is possible to use materials that are relative thin compared to extruded materials while still having odor-proof properties. The sheet walls can be for example welded or glued together, or attached in any other way known to the person skilled in the art.

In one embodiment the first and second sheet wall can be a polymer foil sheet. In order to provide different characteristic to the walls the first and second sheet wall can comprise a layered structure of different sheet materials. For example the outer layer of at least one of the first and second sheet wall is a non-woven material. This provides for an outer layer that is comfortable to the touch and suitable to be arranged along a limb, such as the leg, of a user. In order to even further improve the comfort of the receiving bag the non-woven material may have moisture-dispersing properties. This gives a comfortable and dry feel; especially as a wet feel might unnecessarily have the user worry that a leak has occurred.

In order to handle the receiving bags they may be provided with different handling features, for example ears, which are small protruding flanges that are large enough for a person to hold on to by squeezing a pair of fingers on each side of said flange/ear. In particular as the first distal opening and the second opening are connectable to each other it is disclosed herein that the second proximal opening is provided with a planar coupling member having a handling ear arranged at one side. Thus, the user may easily handle the second receiving bag easily aligning the second proximal opening with the first distal opening.

As mentioned above the second fecal receiving can be arranged on the side of the bed so that a caregiver may monitor it. Thus, the second fecal receiving bag may be provided with at least one through going support hole along its periphery which allows for the second receiving bag to be hung on a hook or similar hanging arrangement. Furthermore a scale is provided on the side of the second fecal receiving device so that the caregiver can see if it is time to empty or change the receiving device. Such a scale can for example be provided with a number of lines having text indicating the level and amount of fecal matter present in the bag at that line.

In order to simply attach the first fecal receiving device around the rectum an adhesive wafer can be attached around the first proximal opening. The adhesive wafer can furthermore be folded back around a folding axis across the adhesive wafer. This allows for an adhesive wafer that is easier to place between the buttocks of a user compare to a planar flat adhesive wafer.

Additionally, as the rectum does not open out perpendicular to the body, i.e. when considering the central axis (vertical direction) extending trough the body created by the intersection of the midsagittal plane and the coronal plane the rectum does not open out in a direction parallel to this central axis. Thus in order to provide for easy application and comfortable wear the adhesive wafer can be attached to the first fecal receiving bag at an angle different from 0° (i.e. parallel) between the center axis of the first proximal opening and the flow direction. It has shown choosing an angle of 35° can accommodated a large group of users.

In order to provide comfort and support and furthermore reduce the risk that the first fecal receiving bag is pulled off, the first fecal receiving bag is provided with a leg strap which supports the bag in its place and distributes some of the stress from the first proximal opening to the strap.

In order to prevent kinking, in particular of the elongated section, the elongated and alternatively part of the reservoir section can be provided with at least a first and a second elongated support strips extending along the longitudinal extent of the elongated section. The strips supports and maintains the shape of the elongated section and reduces the risk that it become bent or twisted.

The elongated support strips may moreover be formed of a resilient material. This allows for a comfortable feel for example if the user lies on the elongated section while it will return to its original state when the user moves away.

The elongated support strips may be provided in many different ways, for example on the outside of the elongated section, within separate compartments or they may be arranged within the lumen of the elongated section. Where they are provided within the lumen a simple construction may be provided wherein a single continuous welding process may create the second fecal receiving bag.

When the elongated section is arranged on e.g. on a linen on the surface of a bed it should move around as little as possible in order to prevent kinking and twisting. By attaching the elongated support strips on the part of the elongated section which touches the surface of the bed they tend to move less around than if one or both were arranged on the opposite side (not lying on the bed surface). Thus, the elongated support strips may be arranged on one bisection of the elongated section. It should be understood that a bisection is the section you obtain when an element is cut or divided into two equal or nearly equal parts, e.g. one of the sheets if the fecal receiving bag is formed of two sheet welded together.

In order to allow gasses to escape and thus avoiding ballooning of the receiving bags the first fecal receiving bag may comprise a filter arrangement allowing gas to escape the first fecal receiving bag. Furthermore, as the first fecal receiving bag also is adapted to wear while walking and standing up the filter arrangement may be placed in proximity of the first proximal opening. This avoids that the fecal matter closes of the filter arrangement, as the fecal matter will move towards the closable first distal opening.

### BRIEF DESCRIPTION OF THE DRAWINGS

FIG. 1 shows a first fecal receiving bag as used in a fecal management system as disclosed herein,

FIG. 2 shows a second fecal receiving bag as used in a fecal management system as disclosed herein,

FIG. 3a-3c show in cross section the elongated section of the second fecal receiving bag,

FIG. 4-7 show a user wearing the first fecal receiving bag, and

FIGS. 8 and 9 show the fecal management system in use.

### DETAILED DESCRIPTION

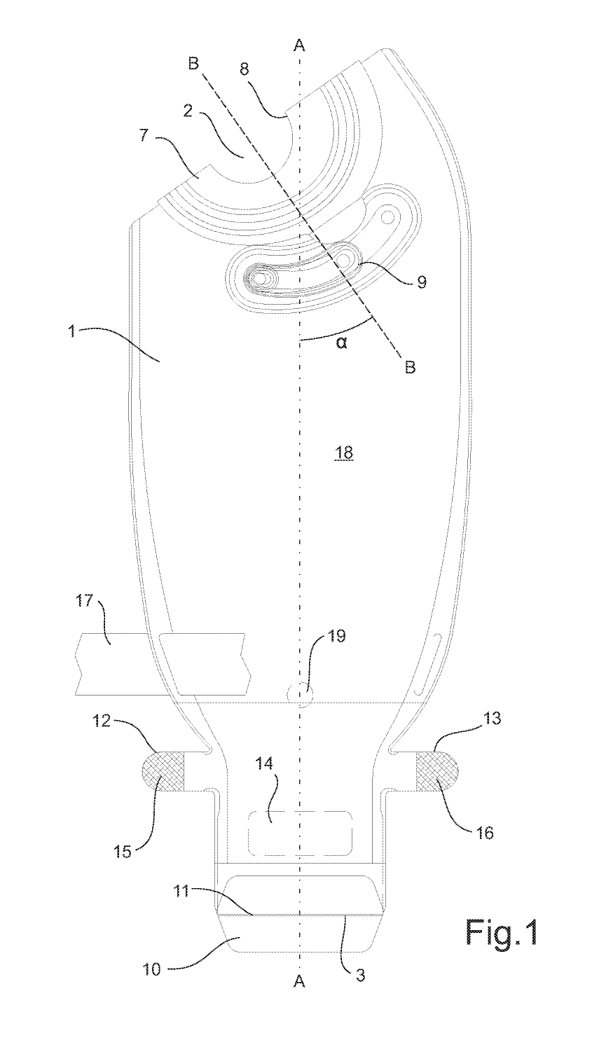

A first fecal receiving bag 1 is shown in FIG. 1. The first bag extends between a first proximal opening 2 and a first distal opening 3 in a flow direction indicated by the solid arrow and which is parallel to the general axis A-A of the first fecal receiving bag. The bag is formed of a first proximal sheet wall 4 and a first distal sheet wall 5 (not seen in FIG. 1). The two sheet walls are welded together along their periphery along first weld 6.

An adhesive wafer 7 is attached to the first fecal receiving bag 1 and is arranged around the first proximal opening 2 having a center axis B-B. A first through going hole 8 is formed in the adhesive wafer allowing communication in or out of the first bag through the first through going hole 8 and the first proximal opening 2.

The adhesive wafer may be formed of a hydrocolloid adhesive much like the ones used for ostomy appliances that are very skin friendly and adhere well to the surface of the skin.

The adhesive wafer is bent around a symmetric axis and is folded around so that the symmetrical halves touches or almost touches each other. This provides an adhesive wafer that is well suited for adhering around the rectum as it is easily inserted between and adhered to the buttocks. The adhesive wafer is attached around the first proximal opening 2. In order to provide a comfortable fit when worn the adhesive wafer 7 may be arranged on the first fecal receiving bag so that an angle α is formed between the general axis A-A and the center axis B-B of the first proximal opening which is different from 0° (i.e. not parallel). In the embodiment shown it is 35°, however it may be between 0° and 90°, in particular between 30° and 40°.

A filter arrangement 9 is provided in the proximal sheet wall 4 close to the adhesive wafer. The filter arrangement allows gasses to escape from the first fecal receiving bag while at the same time cleaning the gasses for odors. Filter arrangement known within ostomy appliances that fulfill the same requirements may be used for this purpose.

A first planar coupling member 10 is attached to the first fecal receiving bag 1 and is arranged around the first distal opening 3. A second through going hole 11 is formed in the first planar coupling member allowing communication in or out of the first bag through the second through going hole 11 and the first distal opening 3.

The first planar coupling member 10 allows the first fecal receiving bag to be closed and opened in order to empty the receiving bag. Such feature in relation to the present construction is known as Hide-away® outlet produced by Coloplast A/S and which is described in WO 99/66859. Additionally, further utility of the first planar coupling member may be provided, as the planar coupling member also can function as a coupling element coupling first receiving bag with another receiving bag as will be described.

When functioning as a closure the first planar coupling member is folded around the elongated opening and rolled up so that it is aligned with first and second locking strip 12 and 13. Such folding also moves the first Velcro® loop patch 14, which is arranged on the distal sheet wall up in alignment with the first and second locking strips. On the first and second locking strip 12, 13 there is arranged first and second Velcro® hook patches 15, 16 respectively. As the first Velcro® loop patch 14 is folded into alignment with the locking strips, the locking strips are folded around and attached to the Velcro® loop patch by the first and second Velcro® hook patches and thus, the first fecal receiving bag may be held in a closed configuration.

A leg strap 17 is attached to the first fecal receiving bag. When the first fecal receiving bag is attached around the rectum the leg strap is attached around the thigh of the user. This holds the bag in place so that it does not dangles and swings around uncontrollably which causes discomfort to the user. Furthermore, the leg strap also serves to relieve some of the stress that would otherwise be exerted on the adhesive wafer. Excess stress on the adhesive wafer may result in that the adhesive wafer detaches partly or fully from the skin around the rectum.

The sheet walls 4,5 may be partly (shown in the figures) or fully covered by an outer non-woven layer 18. This provides a pleasant feel and thus does not irritate the user as the sides of the first fecal receiving bag often will abut against the skin of the legs.

The folded and closed first planar coupling member may be folded further up and hidden under the outer non-woven layer 18. It is held in place by attaching the first Velcro® loop patch 14 to a third Velcro® hook patch 19 arranged partly under the non-woven layer. From the above it can be understood that the first Velcro® loop patch 14 has a size which allows the first, second and third Velcro® hook patches to be attached thereto.

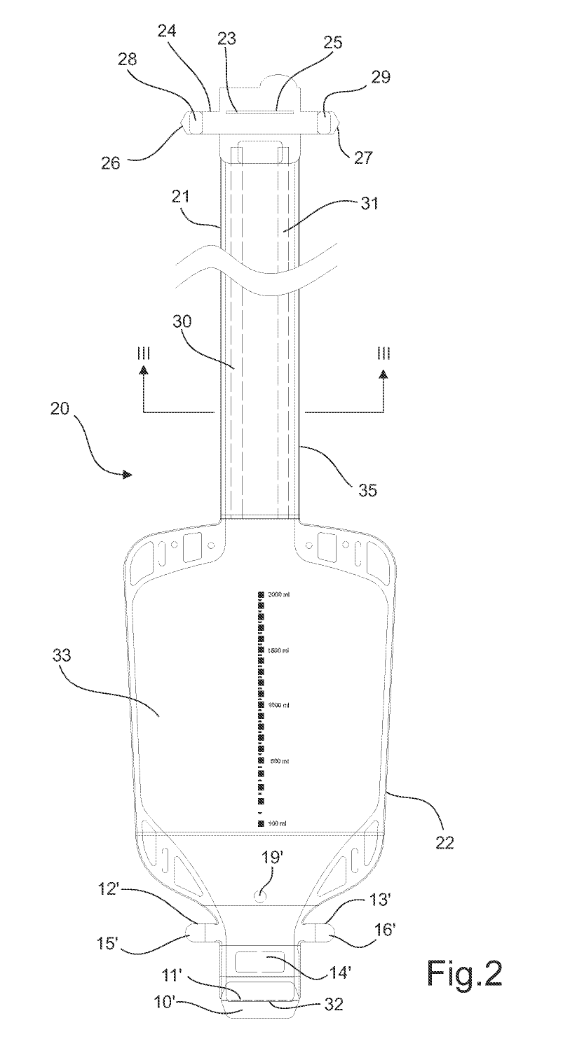

A second fecal receiving bag 20 is shown in FIG. 2. The second fecal receiving bag is formed of an elongated section 21 and a reservoir section 22.

The elongated section 21 extends from a second proximal opening 23 to the reservoir section 22. A second planar coupling member 24 is arranged around the second proximal opening and a third through going hole 25 formed in the second planar coupling member allowing communication in or out of the second fecal receiving bag through the third through going hole 25 and the second proximal opening 23.

The second planar coupling member serves to couple the first and second receiving bag together in a leak and odor tight coupling arrangement. This is done by aligning the second and third through going holes folding the first and second planar coupling member around the second and third through going hole. The coupling members are then secured in this arrangement by folding third and fourth locking strips 26, 27 respectively around the planar coupling members and attaching them to the back of the second planar coupling member via fourth and fifth Velcro® hook patches 28, 29.

This type of coupling arrangement provides a flat and secure, i.e. leak and odor tight, connection between the two receiving bags. However, other coupling arrangements may be anticipated.

The elongated section serves as an extension element to allow the reservoir section to be placed away from the first fecal receiving bag while still being in communication hereto. It can be understood that the first fecal receiving bag and the receiving section may serve as containers for storing and collecting fecal matter and thus have a large capacity relative to the elongated section which serves as an intermediate conveying element. Thus, the construction and placement of the elongated section will often make it prone to kinking, twisting and other effects that cut off the flow of fecal matter.

In order to reduce the risk such cut-off of flow a first and second support strip 30, 31 is arrangement within the elongated section. These strips run within the elongated section for the majority of the extent between the second proximal opening and the reservoir section. The support strips will keep the walls of the elongated section apart allowing better flow while also reducing the risk that the elongated section is twisted and/or bent into a kink.

During use the elongated section may be squeezed by the leg of an user or a caregiver or the user may want to force fecal matter through the elongated section. In order to allow for compression of the elongated section to provide comfort to a user or allow as much fecal matter as possible to be squeezed through the support strips may be formed of a compressible material. Preferably they may be formed of a resilient material which after compression allows it to return to its initial expanded configuration.

The elongated section communicates with the reservoir section, which extends from the elongated section to a second distal opening 32. A closable arrangement similar to that around the first distal opening 3 of the first fecal receiving bag is provided around the second distal opening 32. Thus, the second fecal receiving bag may be emptied when full. Like reference numbers followed by a mark (′) has been used for the element forming the closable arrangement that are similar to that of the first fecal receiving bag.

Accordingly a third planar coupling member 10′ is arranged around the second distal opening 32. A third through going hole 11′ is formed in the third planar coupling member allowing communication with the reservoir section. In order to close of the second distal opening fifth and sixth locking strips 12′, 13′; second Velcro® loop patch 14′; and sixth, seventh and eight Velcro® loop patches 15′,16′,19′ has been provided.

Similar to the first fecal receiving bag the second fecal receiving bag is also formed of a second proximal sheet wall 33 and a second distal sheet wall 34 (not seen in FIG. 2). The two sheet walls are welded together along their periphery along second weld 35.

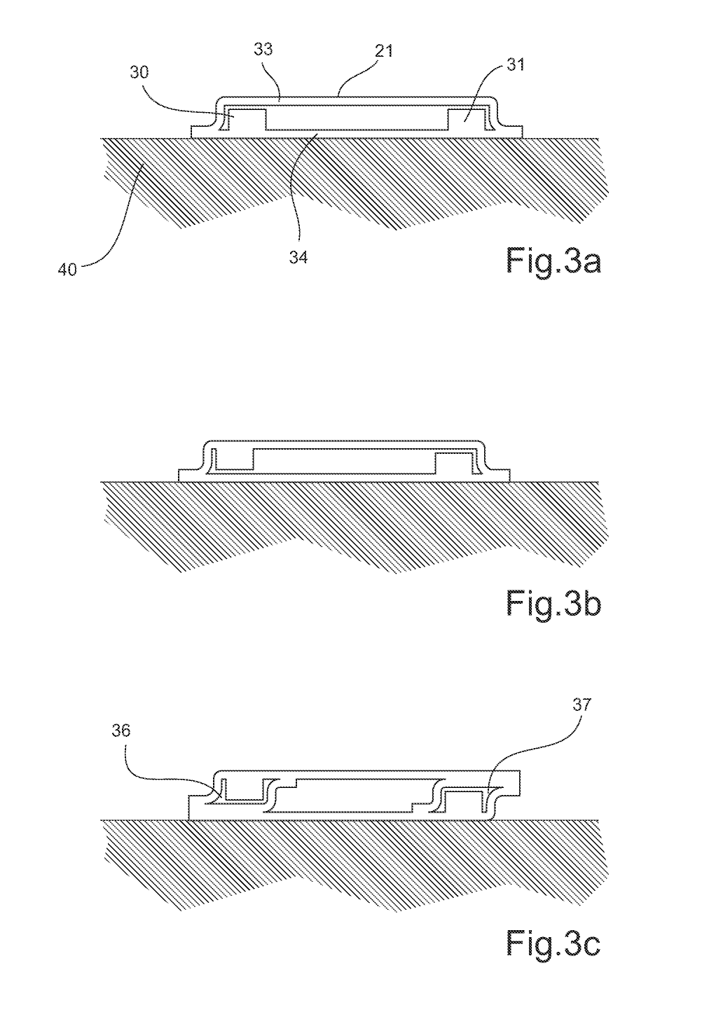

A cross section along the line III-III in FIG. 2 is shown in FIG. 3a. As can be seen the elongated section 21 is formed of the second proximal sheet wall and the second proximal sheet wall that are welded together. First and second support strips 30, 31 are attached to the second distal sheet wall 34 on the surface inside the lumen of the elongated section. The support strips can be attached in many ways, for example welding or gluing.

The elongated section is arranged so that the second distal sheet wall lies on the surface of a bed 40. This provides a solution where the risk of the elongated section is reduced as the support strips will not slide back and forth since they are frictional engaged to the surface of the bed via the second distal sheet wall.

Alternative embodiments of the arrangement of the first and second support strip can be seen in FIG. 3b where the first support strip is arranged on the second proximal sheet wall and the second support strip is arranged on the second distal sheet wall; and in FIG. 3c where the support strips are arranged inside first and second separate channels 36, 37 running parallel with the elongated section.

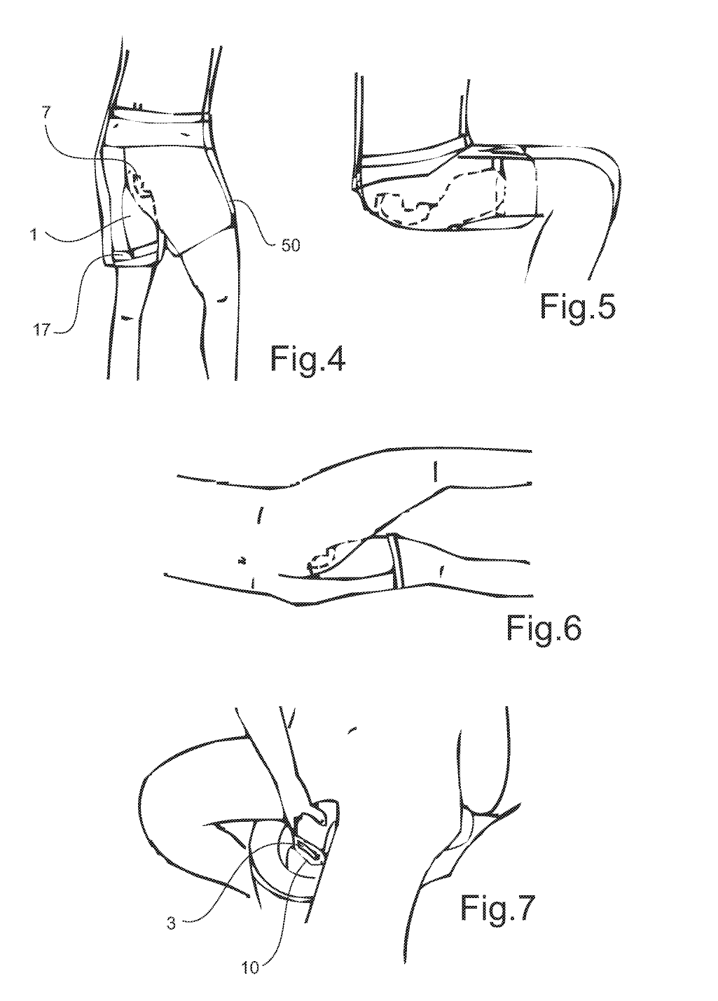

FIGS. 4-7 shows different situation wherein a user is using a first fecal receiving bag 1. The first fecal receiving bag is formed so that it can be worn on the inside of the thigh of a user. Thus, different sizes of bags may be required for different people. However, it has shown that a bag having a length of 270 mm and a width of 130 mm will satisfy a majority of people. Although these dimension provides a total volume of 780 ml the buttocks will squeeze the upper area around the adhesive wafer so that the volume is reduced to an effective 300 ml. However, 300 ml is sufficient to allow a user to move around for an extended period time, from a few hours up to a day, without having to worry about changing or emptying the first fecal receiving bag.

FIG. 4 shows a user who is walking around. As can be seen the first fecal receiving bag 1 fits nicely under a pair of shorts 50 or other garment such as a dress or pair of pants. The leg strap 17 holds the bag in place along the thigh avoiding that the bag starts swinging out of rhythm with the users walking pace. At the same time the leg strap support the first fecal receiving bag taking some of the stress that would otherwise be exerted on the adhesive wafer 7.

As the user sits down, which can be seen in FIG. 5, the bag will follow orientation of the thigh and be kept between the legs in a comfortable position where the user does not risk sitting on it or it gets squeezed between objects.

Furthermore as the user lies down as shown in FIG. 6 the receiving bag will lie safely between the legs avoiding that it gets squeezed between the user and the bed as the user turns during the night.

FIG. 7 shows how the user easily empties the first fecal receiving device. Before he sits down on the he removes the leg strap. When seated he can then open the first distal opening 3 and by stroking his hand along the sides of the first fecal receiving bag the fecal matter contained in the bag will be squeezed out. Using a piece of toilet paper he can then easily clean the first planar coupling member 10 and close the first distal opening 3.

When users go to sleep or who are bedridden need to get up during the night or have caring personnel come and help them empty the fecal receiving bag. In such cases a second fecal receiving device be may attached to the first fecal receiving device as an extension member. The second fecal receiving device has a capacity that is much larger than the first fecal receiving device, e.g. 2 litres. This allows for a user to have a full nights sleep and caregivers does not have to attend the user as often.

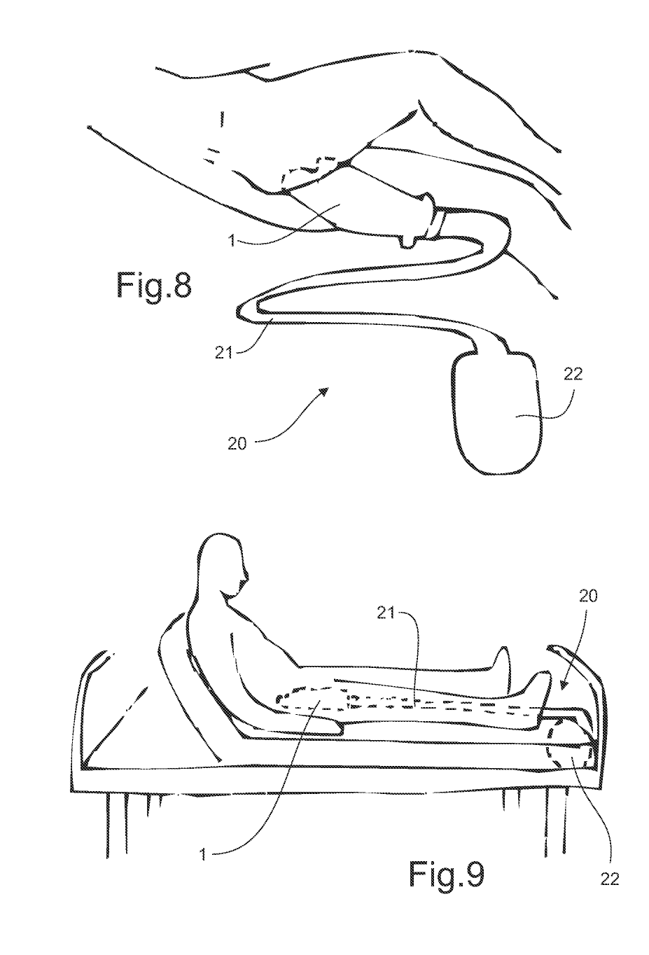

As can be seen from FIG. 8 such second fecal receiving bag may be connected to the first fecal receiving bag via an extension section. As shown in FIG. 9 such extension section allows the second fecal receiving device to be hung on the side of the bed so that caring personal may easily monitor the volume of fecal matter in the second bag without having to trouble user. When the second fecal receiving bag is full the whole arrangement, e.g. both the first fecal receiving bag and the second fecal receiving, may be exchanged or only the second fecal receiving bag may be exchanged. Alternatively, or additionally, a closable opening may also be provided on the second fecal receiving bag allowing it to be emptied without detaching it from the first fecal receiving bag.

### REFERENCE NUMBERS

1. first fecal receiving bag
2. first proximal opening
3. first distal opening
4. proximal sheet wall
5. distal sheet wall
6. weld
7. adhesive wafer
8. first through going hole
9. filter arrangement
10. first planar coupling member
11. second through going hole
12. first locking strip
13. second locking strip
14. first Velcro® loop patch
15. first Velcro® hook patch
16. second Velcro® hook patch
17. a leg strap
18. outer non-woven layer
19. third Velcro® hook
20. second fecal receiving bag
21. elongated section
22. reservoir section
23. second proximal opening
24. second planar coupling member
25. third through going hole
26. third locking strip
27. fourth locking strip
28. fourth Velcro® hook patch
29. fifth Velcro® hook patch
30. first support strip
31. second support strip
32. second distal opening
33. second proximal sheet wall
34. second distal sheet wall
35. second weld
36. first separate channel
37. second separate channel

1. A fecal management system for collecting fecal matter from the rectum, comprising a first fecal receiving bag extending in a flow direction between a first proximal opening attachable around the rectum and a closable first distal opening, said fecal management system further comprises a second fecal receiving bag having an elongated section extending in the flow direction between a second proximal opening and a reservoir section, said reservoir section extending in the flow direction between a proximal end attached to the elongated section and a distal end, wherein the first distal opening is connectable to the second proximal opening.

2. A fecal management system according to claim 1, wherein the first fecal receiving bag and the second fecal receiving bag are each formed of a first sheet wall and a second sheet wall attached to each other along their periphery.

3. A fecal management system according to claim 2, wherein the first and second sheet walls comprise a polymer foil sheet.

4. A fecal management system according to claim 2, wherein the first and second sheet walls are each fabricated from a layered structure of different sheet materials.

5. A fecal management system according to claim 2, wherein an outer layer of at least one of the first and second sheet walls is a non-woven material.

6. A fecal management system according to claim 5, wherein the non-woven material has a moisture dispersal property.

7. A fecal management system according to claim 2, wherein the first and second sheet walls are formed of non-elastic materials.

8. A fecal management system according to claim 1, wherein the second proximal opening is provided with a planar coupling member having a handling ear arranged at one side.

9. A fecal management system according to claim 1, wherein the second fecal receiving bag forms at least one through going support hole on its periphery.

10. A fecal management system according to claim 1, wherein a volume measurement scale is provided on a side of the second fecal receiving bag.

11. A fecal management system according to claim 1, wherein an adhesive wafer is attached around the first proximal opening, the adhesive wafer folded on a folding axis to provide an exposed exterior surface of adhesive configured to adhere around the anus between buttocks of a wearer.

12. A fecal management system according claim 11, wherein the folding axis forms an angle of less than 90° relative to the flow direction.

13. A fecal management system according to claim 12, wherein the angle is 32°.

14. A fecal management system according to claim 1, wherein the first fecal receiving bag includes a leg strap.

15. A fecal management system according to claim 1, wherein the elongated section is provided with a first elongated support strip spaced apart from a second elongated support strips, the elongated support strips disposed inside of the elongated section and extending along a portion of a longitudinal extent of the elongated section.

16. A fecal management system according to claim 15, wherein the elongated support strips are formed of a resilient material.

17. A fecal management system according to claim 15, wherein the elongated support strips are spaced apart and disposed along opposing longitudinal seams of the elongated section.

18. A fecal management system according to claim 15, wherein the elongated support strips are arranged on a backing wall of the elongated section.

19. A fecal management system according to claim 1, wherein the first fecal receiving bag comprises a filter arrangement allowing gas to escape the first fecal receiving bag.

20. fecal management system according to claim 19, wherein the filter arrangement is placed in proximity of the first proximal opening.

21. A fecal management system according to claim 1, wherein a closable second distal opening is provided at the distal end.
